(19) United States
(12) Patent Application Publication
(10) Pub. No.: US 2023/0298720 A1
(43) Pub. Date: Sep. 21, 2023

(54) DEEP LEARNING MODEL PREDICTION METHOD OF DRUG IC50 BASED ON MOLECULAR STRUCTURE AND GENE EXPRESSION

(71) Applicants: Precision Scientific Co., Ltd., Beijing (CN); Precision Scientific Biomedicine (Suzhou) Co., Ltd., Suzhou (CN); Precision Scientific (Beijing) Co., Ltd., Beijing (CN)

(72) Inventors: Xuwo JI, Beijing (CN); Xinxin PENG, Beijing (CN); Danyang YU, Beijing (CN)

(21) Appl. No.: 18/091,965

(22) Filed: Dec. 30, 2022

(30) Foreign Application Priority Data
Mar. 21, 2022 (CN) 202210275508.7

(51) Int. Cl. G16H 20/10; G06F 18/214; G06N 3/08; G06Q 10/04; G16B 50/30

(52) U.S. Cl. CPC G16H 20/10; G06F 18/214; G06N 3/08; G06Q 10/04; G16B 50/30

(57) ABSTRACT

A drug IC50 deep learning model prediction method based on molecular structure and gene expression includes establishing a deep learning model to predict drug IC50 in different cell lines; predicting the drug IC50 in different cell lines based on the deep learning model. Also disclosed are prediction systems, electronic devices and computer readable storage media, which use grammar variational autoencoder to encode the chemical molecular formula of drugs and use autoencoder to encode cell line expression data, predict the drug IC50 in different cell lines through neural network methods, and predict the drug IC50 values of drugs in different types of cancer cell lines directly through the molecular information of drugs, which can reduce the investment of funds and time in preclinical development. Applying the model to patients helps screen out the applicable population of drugs, reduces unnecessary clinical trials, and improves the success rate of clinical trials.

### TECHNICAL FIELD

The invention relates to the field of medical information technology, in particular to a prediction method of drug IC50 deep learning model, system, electronic equipment and computer readable storage medium based on molecular structure and gene expression.

### BACKGROUND OF THE PRESENT INVENTION

According to the survey, the current average cost of developing a new drug is 1.359 billion US dollars, and the average research and development time is 12 years. It can be seen that developing a new drug requires a lot of capital and time costs. It is one of the effective ways to reduce the cost of R&D investment to find new indications for drugs that have been marketed or have completed part of the R&D process. However, the action mechanism of drug molecules is very complex, and the effects in different cells, especially in different cancer cells, are also different. Therefore, studying the role of drugs in different cancer cells usually requires expensive, time-consuming and expensive biological experiments. In the existing technology, we need to obtain the IC50 values of drugs in different cell lines through cell line experiments (IC50 refers to the concentration of drugs required when the number of cells is reduced by half compared with control. IC50 values can be used to measure the ability of drugs to cause cancer cell apoptosis, that is, the stronger the ability to cause cancer cell apoptosis, the lower the value is, which can also indicate the tolerance of a certain cell to drugs in reverse). Obtaining the IC50 value of a drug in a cancer cell line requires multiple experiments. However, we currently have thousands of cancer cell lines, and it is very difficult to collect and purchase these cell lines. To obtain the IC50 values of hundreds of drugs in these cell lines, hundreds of thousands of experiments are further needed, which will cost a lot of manpower, material resources, financial resources and time.

With the development of machine learning, especially machine learning model or deep learning technology, more and more scientific problems can be solved through deep learning. First of all, basic primary calculation methods are used to predict IC50 to reduce input. For example, the technical solution disclosed in the article “Deep Generation Neural Network for Accurate Prediction of Drug Response Filling” published in Nature Communications only includes the evaluation for the accuracy of the training set, and the effect is limited. Only 50.65% of drugs have a correlation coefficient between predicted IC50 and the real drug lethal dose greater than 0.5.

In addition, at present, the IC50 value drug experiment of the sample cannot be directly applied to the patient tissue samples, and it is unable to accurately predict the patient’s response to the drug. Therefore, the calculation method is needed to predict the response of patients to drugs through the expression profile of patient tissues, so as to screen the effective population of drugs, increasing the complexity of the prediction scheme.

Therefore, it can be said that there is no complete solution in the existing technology that effectively combines the in-depth learning method with drug development and biological experiment to solve the problem of accurately predicting the IC50 of drug molecules in different cell lines, especially cancer cell lines.

### SUMMARY OF THE PRESENT INVENTION

In order to solve the problems in the prior art, the present invention provides the following technical solution, which uses the grammar variational autoencoder to encode the chemical formula of the drug and the autoencoder to encode the cell line expression data, and predicts the IC50 of the drug in different cell lines through the neural network method.

In one aspect, the invention provides a deep learning model for the drug IC50 prediction based on molecular structure and gene expression, including:
S1, establishing a deep learning model to predict the drug IC50 in different cell lines;
S2, predicting the drug IC50 in different cell lines based on the deep learning model.

Further, the cell line is a cancer cell line.

Further, S1, establishing a deep learning model to predict the drug IC50 in different cell lines, comprising:
S11, obtaining the samples for establishing the deep learning model, and preprocessing the samples to obtain sample data; and
S12, constructing the deep learning model.

Further, S11 comprising:
S111, downloading data of cell line expression profile from related cell line database; In the meantime, downloading the drug IC50 in different cell lines from the drug sensitivity genomics database;
S112: cleaning up the data of the cell line expression profile and the IC50 value, including: in the data of the cell line expression profile, retaining the genes with average expression value being greater than the first threshold in all cell lines; deleting the drug data of all drugs corresponding to the drug IC50 that cannot use rdkit and/or the drug data that cannot be read by the grammar variational autoencoder (GVAE); the cleaned data of the cell line expression profile and the cleaned drug IC50 values constitute the data of the deep learning model.

Further, the first threshold value can be selected from 0.5-2, preferably 1.

Further, the S12 comprising:
S121, training the deep learning model, the training including one or more rounds, and each round of the training including:
   (1) randomly selecting 80% of the sample data from the sample data as the training set, and 20% of the sample data as the test set. The training set and the test set being used for the training and evaluation of the depth learning model;
   (2) encoding the chemical formula of the drug based on the simplified molecular input line input system and weight file in the grammar variational autoencoder to obtain a 56-dimensional feature vector to represent the molecular information of the drug;
   (3) based on the cleaned expression profile of the cell line and the autoencoder, reading the expression profile data of the cell line, and obtaining the n-dimensional cell line feature vector to represent the cell line, with the range of n being 50-150;
   (4) establishing the basic model of the deep learning model, wherein the 56-dimensional feature vector and the n-dimensional cell line feature vector are used as the input of the basic model, the predicted value of drug IC50 is used as the output, and the basic model uses 2-6 layers of fully connected neural network, preferably 4 layers;
   (5) taking cosine similarity or Pearson correlation coefficient and minimum mean square error as objective optimization function, using Adam optimizer as descent method, and use the data in the training set to train the depth learning model;
S122, model effectiveness validation, including:
   validating the effectiveness of the model based on the data in the training set and the test set. If the Pearson correlation coefficient between the real drug IC50 in the training set and the predicted lethal dose of drugs is greater than the second threshold, and the Pearson correlation coefficient between the real drug IC50 in the test set and the predicted lethal dose of drugs is greater than the third threshold, and then proceeding to step S123;
S123, based on the training and the validation of the model effectiveness, obtaining the deep learning model.

Further, the S122 further includes:

selecting the gene expression profile and curative effect data in the database; If the Pearson correlation coefficient between the predicted value of drug IC50 in cancer cells of patients and the tumor reduction ratio of patients using specific elements being greater than the fourth threshold, and the correlation coefficient with the survival time of patients is less than the fifth threshold, it proves that the deep learning model is effective; and/or selecting the gene expression profile and curative effect data in the database. If the IC50 value predicted by the model in patients with incomplete tumor disappearance is greater than that predicted by the model in patients with complete tumor disappearance, the deep learning model being proved to be effective.

In the second aspect of the invention, a prediction system of drug IC50 deep learning model based on molecular structure and gene expression is provided, including:
a deep learning model establishing module, being used to establish a deep learning model to predict the drug IC50 in different cell lines;
an drug IC50 prediction module, being used to predict the drug IC50 in different cell lines based on the deep learning model.

A third aspect of the present invention provides an electronic device, including a processor and a memory, wherein the memory stores a plurality of instructions, and the processor is used to read the instructions and execute a method as described in the first aspect.

A fourth aspect of the present invention provides a computer-readable storage medium, wherein the computer-readable storage medium stores a plurality of instructions, which can be read by a processor and executed by the method described in the first aspect.

The prediction method, system and electronic device of drug IC50 deep learning model based on molecular structure and gene expression provided by the invention have the following beneficial effects:

In this invention, the grammar variational autoencoder is used to encode the chemical formula of the drug and the autoencoder is used to encode the cell line expression data. The drug IC50 in different cell lines can be predicted by the neural network method. The drug IC50 in different types of cancer cell lines can be predicted directly through the molecular information of the drug, which can reduce the capital and time investment in preclinical development to a certain extent. Applying the model to patients can help screen out the applicable population of drugs, reduce unnecessary clinical trials, and thus improve the success rate of clinical trials.

### BRIEF DESCRIPTION OF THE DRAWINGS

FIG. 1 is a flow diagram of the prediction method of drug IC50 deep learning model based on molecular structure and gene expression described in the invention.

FIG. 2 is the schematic diagram of the prediction system of drug IC50 deep learning model based on molecular structure and gene expression provided by the invention.

FIG. 3 is a structural diagram of an embodiment of the electronic device provided by the invention.

### DETAILED DESCRIPTION OF THE PRESENT INVENTION

In order to better understand the above technical solution, the following will give a detailed description of the above technical solution in combination with the drawings of the description and specific embodiments.

The method provided by the invention can be implemented in the following terminal environment, and the terminal can include one or more of the following components: processor, memory and display screen. At least one instruction is stored in the memory, and the instruction is loaded and executed by the processor to realize the method described in the following embodiment.

A processor may include one or more processing cores. The processor uses various interfaces and lines to connect various parts of the entire terminal, and executes various functions and processing data of the terminal by running or executing instructions, programs, code sets or instruction sets stored in memory, and calling data stored in memory.

Memory can include random access memory (RAM) or read only memory (ROM). Memory can be used to store instructions, programs, codes, code sets, or instructions.

The display screen is used to display the user interface of each application.

In addition, it can be understood by those skilled in the art that the structure of the above terminal does not define the terminal, and the terminal can include more or fewer components, or combination of some components, or different component arrangements. For example, the terminal also includes RF circuit, input unit, sensors, audio circuits, power supply and other components, which will not be described here.

### Example 1

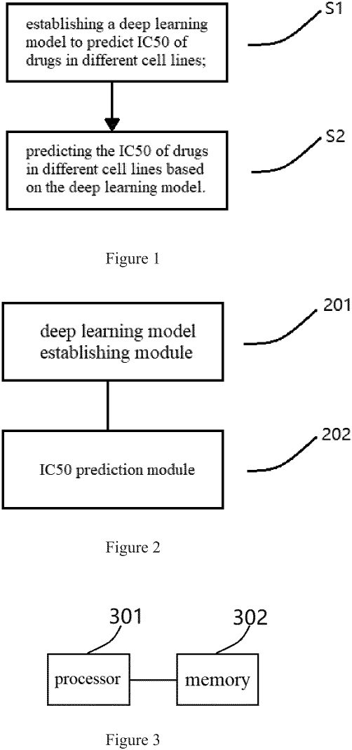

As shown in FIG. 1, this embodiment provides a drug differential expression profile and indication prediction method based on a deep learning model, which is specifically used for the drug IC50 prediction in the context of cancer cell lines, including:
S1, establishing a deep learning model to predict the drug IC50 of in different cancer cell lines;
S2, predicting the drug IC50 in different cancer cell lines based on the deep learning model.

Further, the software dependency environment used in this embodiment is python3.7, Keras2.3.0, tensorflow-gpu1.15.0, rdkit2021.03.5, and step S1 includes:
S11, obtaining the samples for establishing the deep learning model, and preprocessing the data; include:
S111, downloading the data of cell line expression profile from the Cancer Cell Line Encyclopedia database; in the meantime, downloading the drug IC50 in different cell lines from the Genomics of Drug Sensitivity in Cancer database;
S112: cleaning up the data of the cell line expression profile and the drug IC50 value, including: in the data of the cell line expression profile, retaining the genes with average expression value being greater than the first threshold in all cell lines; deleting the drug data of all drugs corresponding to the drug IC50 value that cannot use rdkit and/or the drug data that cannot be read by the grammar variational autoencoder (GVAE); the cleaned data of the cell line expression profile and the cleaned drug IC50 value constitute the data of the deep learning model.

S12, constructing the deep learning model, comprising:

S121, training the deep learning model, the training including one or more rounds, and each round of the training including:
(1) randomly selecting 80% of the sample data from the sample data as the training set, and 20% of the sample data as the test set. The training set and the test set being used for the training and evaluation of the depth learning model;
(2) encoding the chemical formula of the drug based on the simplified molecular input line input system (SMILES) and weight file zinc_ vae_ grammar_ L56_E100_val in the grammar variational autoencoder (GVAE) to obtain a 56-dimensional feature vector to represent the molecular information of the drug;
(3) based on the cleaned expression profile of the cell line and the autoencoder, reading the expression profile data of the cell line, and obtaining the n-dimensional cell line feature vector to represent the cell line, with the range of n being 50-150, and preferably 100;
(4) establishing the basic model of the deep learning model, wherein the 56-dimensional feature vector and the n-dimensional cell line feature vector are used as the input of the basic model, the predicted value of drug IC50 is used as the output, and the basic model uses 2-6 layers of fully connected neural network, preferably 4 layers; In this embodiment, the basic model uses a 4-layer fully connected neural network; The neural network comprises an input layer, a first layer, a second layer, a third layer and a fourth layer, and the specific parameters are as follows:
   Input layer: number of nodes 156;
      Layer 1: The number of nodes can be selected from 256 to 2048. The activation function is ReLu and the dropout ratio can be selected from 0.1 to 0.3;
      Layer 2: The number of nodes can be selected from 256 to 2048, the activation function is ReLu and the dropout ratio can be selected from 0.1 to 0.3;
      Layer 3: The number of nodes can be selected from 256 to 2048, the activation function is ReLu and the dropout ratio can be selected from 0.1 to 0.3;
      Layer 4: The number of nodes is 1, and the activation function is linear.
(5) Take cosine similarity as the objective optimization function, use Adam optimizer as the descent method, and use the data in the training set to train the depth learning model;

Among them, the optional range of the training batch size is 56-512, and the optional range of x is 32-256 when training x rounds using the data in the training set.

S122, model effectiveness validation, including:

selecting the gene expression profile and curative effect data in the database; If the Pearson correlation coefficient between the predicted the drug IC50 in cancer cells of patients and the tumor reduction ratio of patients using specific elements being greater than the fourth threshold, and the correlation coefficient with the survival time of patients is less than the fifth threshold, it proves that the deep learning model is effective; and/or selecting the gene expression profile and curative effect data in the database. If the drug IC50 predicted by the model in patients with incomplete tumor disappearance is greater than that predicted by the model in patients with complete tumor disappearance, the deep learning model being proved to be effective.

In this preferred embodiment, the fourth threshold value is 0.2, and the fifth threshold value is - 0.3. Of course, those skilled in the art can select different threshold points or threshold ranges as required, which are all within the protection scope of the application.

S123, based on the training and model effectiveness validation, to obtain a deep learning model.

In this embodiment, the effectiveness of the model is validated by using the gene expression profile and curative effect data in the number GSE66305 and GSE50509 database of gene expression comprehensive database. In GSE66305, the Pearson correlation coefficient between the predicted drug IC50 in cancer cells of patients and the tumor reduction ratio of patients using Dalafinil is 0.28, and the correlation coefficient with the survival time of patients is -0.37. In GSE50509, the average drug IC50 in cancer cells predicted by the model in patients with completed tumor disappearance is 1.87; In patients with non-completed tumors disappearance, the drug IC50 in cancer cells predicted by the model is 2.02. Both data sets prove that the drug IC50 predicted by the model can reflect the actual drug effect for patients to a certain extent.

### Example 2

As shown in FIG. 2, this embodiment provides a prediction system of drug IC50 deep learning model based on molecular structure and gene expression is provided, including:
a deep learning model establishing module 201, being used to establish a deep learning model to predict drug IC50 in different cell lines; and
an IC50 prediction module 202, being used to predict the drug IC50 in different cell lines based on the deep learning model.

The system can realize the prediction method provided in Example 1 above. The specific prediction method can refer to the description in Example 1, and will not be repeated here.

The invention also provides a memory, which stores a plurality of instructions for implementing the method of Example 1.

As shown in FIG. 3, the invention also provides an electronic device, including a processor 301 and a memory 302 connected to the processor 301. The memory 302 stores multiple instructions, which can be loaded and executed by the processor, so that the processor can execute the method in Example 1.

Although the preferred embodiments of the present invention have been described, those skilled in the art may make additional changes and modifications to these embodiments once they have learned the basic inventive concept. Therefore, the appended claims are intended to be interpreted as including preferred embodiments and all changes and modifications falling within the scope of the present invention. Obviously, those skilled in the art can make various changes and modifications to the invention without departing from the spirit and scope of the invention. Thus, if these modifications and variations of the present invention fall within the scope of the claims of the present invention and their equivalents, the present invention is also intended to include these modifications and variations.

What is claimed is:

1. A deep learning model for drug IC50 prediction based on molecular structure and gene expression, which comprises:
S1, establishing a deep learning model to predict drug IC50 in different cell lines;
S2, predicting the drug IC50 in different cell lines based on the deep learning model;
wherein, S1, establishing a deep learning model to predict the drug IC50 in different cell lines, comprising:
S11, obtaining the samples for establishing the deep learning model, and preprocessing the samples to obtain sample data; and
S12, constructing the deep learning model.
S11 comprising:
S111, downloading data of cell line expression profile from cell line related database; In the meantime, downloading the drug IC50 values of drugs in different cell lines from the drug sensitivity genomics database;
S112: cleaning up the data of the cell line expression profile and the IC50 value, including: in the data of the cell line expression profile, retaining the genes with average expression value being greater than the first threshold in all cell lines; deleting the drug data of all drugs corresponding to the drug IC50 value that cannot use rdkit and/or the drug data that cannot be read by the grammar variational autoencoder (GVAE); the cleaned data of the cell line expression profile and the cleaned IC50 value constitute the sample data of the deep learning model;
S12 comprising:
S121, training the deep learning model;
S122, model effectiveness validation, including:
validating the effectiveness of the model based on the data in the training set and the test set. If the Pearson correlation coefficient between the real drug IC50 in the training set and the predicted lethal dose of drugs is greater than the second threshold, and the Pearson correlation coefficient between the real drug IC50 in the test set and the predicted lethal dose of drugs is greater than the third threshold, and then proceeding to step S123;
S123, based on the training and the validation of the model effectiveness, obtaining the deep learning model;
S122 further includes:
selecting the gene expression profile and curative effect data in the database; If the Pearson correlation coefficient between the predicted drug IC50 in cancer cells of patients and the tumor reduction ratio of patients using specific elements being greater than the fourth threshold, and the correlation coefficient with the survival time of patients is less than the fifth threshold, it proves that the deep learning model is effective; and/or selecting the gene expression profile and curative effect data in the database. If the drug IC50 value predicted by the model in patients with incomplete tumor disappearance is greater than that predicted by the model in patients with complete tumor disappearance, the deep learning model being proved to be effective.

2. A drug IC50 deep learning model prediction method based on molecular structure and gene expression according to claim 1, wherein,
the cell line is a cancer cell line.

3. A drug IC50 deep learning model prediction method based on molecular structure and gene expression according to claim 1, wherein,
the first threshold value can be selected from 0.5-2.

4. A drug IC50 deep learning model prediction method based on molecular structure and gene expression according to claim 1, wherein,
the training in S121 including one or more rounds, and each round of the training including:
(1) randomly selecting 80% of the sample data from the sample data as the training set, and 20% of the sample data as the test set. The training set and the test set being used for the training and evaluation of the depth learning model;
(2) encoding the chemical formula of the drug based on the simplified molecular input line input system and weight file in the grammar variational autoencoder to obtain a 56-dimensional feature vector to represent the molecular information of the drug;
(3) based on the cleaned expression profile of the cell line and the autoencoder, reading the expression profile data of the cell line, and obtaining the n-dimensional cell line feature vector to represent the cell line, with the range of n being 50-150;
(4) establishing the basic model of the deep learning model, wherein the 56-dimensional feature vector and the n-dimensional cell line feature vector are used as the input of the basic model, the predicted drug IC50 is used as the output, and the basic model uses 2-6 layers of fully connected neural network, preferably 4 layers;
(5) taking cosine similarity or Pearson correlation coefficient and minimum mean square error as objective optimization function, using Adam optimizer as descent method, and use the data in the training set to train the depth learning model.

5. A prediction system of drug IC50 deep learning model based on molecular structure and gene expression, utilizing to implement the prediction method of drug IC50 deep learning model based on molecular structure and gene expression, which comprises:
a deep learning model establishing module, being used to establish a deep learning model to predict drug IC50 in different cell lines;
an IC50 prediction module, being used to predict the drug IC50 in different cell lines based on the deep learning model.

6. A memory, which comprises storing a plurality of instructions for implementing the prediction method as described according to claim 1.

7. An electronic device, which comprises a processor and a memory connected with the processor, and the memory stores a plurality of instructions, and the instructions can be loaded and executed by the processor, so that the processor can execute the prediction method as described according to claim 1.
